(19) United States
(12) Patent Application Publication
(10) Pub. No.: US 2019/0059782 A1
(43) Pub. Date: Feb. 28, 2019

(54) SYSTEM AND METHOD USING CARDIAC-ESOPHAGEAL IMPEDANCE MAPPING TO PREDICT AND DETECT ESOPHAGEAL INJURY DURING CARDIAC ABLATION PROCEDURES

(71) Applicant: THE METHODIST HOSPITAL SYSTEM, Houston, TX (US)

(72) Inventors: Miguel Valderrabano, Houston, TX (US); Amish S. Dave, Houston, TX (US)

(21) Appl. No.: 16/080,059

(22) PCT Filed: Feb. 28, 2017
(86) PCT No.: PCT/US2017/019877

§ 371 Date: Aug. 27, 2018

Related U.S. Application Data
(60) Provisional application No. 62/301,195, filed on Feb. 29, 2016.

(51) Int. Cl. A61B 5/053; A61B 5/042; A61B 5/0205; A61B 18/14

(52) U.S. Cl. CPC A61B 5/0538; A61B 5/0421; A61B 5/6852; A61B 18/14; A61B 5/0205

(57) ABSTRACT

Exemplified methods and apparatus use assess electrical coupling between an ablative catheter and the esophagus using measurements of electrical impedance to beneficially predict esophageal damage prior to ablation and to detect on-going esophageal damage during ablation. The technology facilitates the determination of regional variations of electrical coupling between the esophagus and the ablation catheter to infer the risk of the esophagus and its nearby structures.

### RELATED APPLICATIONS

This application claims priority to, and the benefit of, U.S. Provisional Application No. 62/301,195, filed “Feb. 29, 2016”, title “System and Method Using Cardiac-Esophageal Impedance Mapping to Predict and Detect Esophageal Injury During Cardiac Ablation Procedures”, which is incorporated by reference herein in its entirety.

### TECHNICAL FIELD

This application is directed to the prevention of esophageal injury, specifically using electric coupling measurements such as impedance.

### BACKGROUND

During cardiac ablation procedures, cardiac tissue (e.g., abnormal tissue or undesired tissue) is destroyed (ablated) by the use of tissue-disrupting energy such as radiofrequency, ultrasound, microwave, cryo-thermal, among others. Because of the proximity of the esophagus to cardiac structures—specifically the left atrium—collateral damage, via ablation, to the esophagus can occur, which may lead to death. This is one of the most feared possible complications of cardiac ablation.

In the United States, in 2013, it is estimated that about 80,000 endovascular ablation operations were performed to treat atrial fibrillation (AF). This number is expected to sharply rise because the number of individuals with heart disease is expected to more than double over the next 20 years. Ablation for atrial fibrillation is a surgical procedure that treats abnormal heart rhythms by creating a series of lesions around the pulmonary vein ostia in the posterior left atrium (LA). The esophagus is separated from the posterior left atrium by a thin (about 0.9±0.2 mm) layer of tissue and fluid in the oblique sinus of the pericardium, and is, therefore, particularly prone to thermal injury during AF ablation.

Fistula (an abnormal connection between organs) can result from thermal injury to the esophagus and is difficult to detect. In certain literatures, the associated mortality rate reported of such injuries is very high (93%), with 13 deaths occurring in 14 reported cases. It has also been reported that in these cases, 50% of patients with an atrio-esophageal fistula were not correctly diagnosed until autopsy. Fistulae occur typically days or weeks after the ablation procedure—not acutely during the procedure—and result from progression of damage to the esophagus that was created at the time of ablation. Aside from the difficulties in making the diagnosis once the fistula occurs, with current technology it is difficult to predict and detect esophageal damage at the time of the ablation procedure, and there are associated risks due to complications with such predictions.

To predict and prevent injury to the esophagus, one type of system employs temperature sensors to detect temperatures of nearby tissues. However, due to thermal inertia, there is lag in time associated with the detection of change in temperature of such tissues. In addition, certain regions in the heart requiring ablation have blind-spots that are not detectable via thermal sensors.

Another type of system employs location sensors mounted to an esophageal catheter. However, such techniques do not provide active feedback of imminent esophageal injury to a surgeon conducting the ablation procedures.

There is a need to more accurately predict and prevent esophageal injury during cardiac ablation procedures.

### SUMMARY

Exemplified methods and apparatuses disclosed herein use assessed electrical coupling between the ablative catheter and the esophagus using measurements of electrical impedance to beneficially predict esophageal damage prior to ablation. The methods and apparatuses also detect on-going esophageal damage during ablation. The methods and apparatuses facilitate the determination of regional variations of electrical coupling between the esophagus and the ablation catheter to inform about the risk of injury to the esophagus and its nearby structures.

The exemplified methods and apparatuses further facilitate 3D mapping of atrio-esophageal impedance, in areas of the heart. The 3D mapping can be expressed in term of impedance parameters or associative risk parameters, to indicate and/or highlight areas of risk, particularly, areas of high risk, during ablation procedures. In addition, the methods and apparatuses may be used to measure tissue integrity during ablation, thus facilitating the operator's modification of the ablation strategy to prevent esophageal damage.

In an aspect, a method for preventing esophageal injury during ablation of tissues in the heart is disclosed. The method includes applying, to a first electrode located within a chamber of the heart of a subject (e.g., a person, an animal, etc.), a first set of one or more electrical signals (e.g., via a current source, wherein the one or more signals have RF frequencies); measuring, from a second electrode located in the esophagus of the subject, a second set of electrical signals resulting from the first set of one or more electrical signals being applied to the first electrode, wherein the second set of electrical signals characterizes an atrio-esophageal electric coupling (e.g., an atrio-esophageal impedance) between the first electrode located in the chamber of the heart and the second electrode located in the esophagus; and in response to the atrio-esophageal electric coupling, or a derivative parameter derived therefrom (e.g., proximity value), satisfying an alert condition (e.g., an impedance or derived proximity value being within an alert condition, or outside a non-alert condition, e.g., based on analog or numerically-derived threshold), causing an audible or visual alert to be generated.

In some embodiments, the method includes determining, by a processor, an atrio-esophageal electric coupling parameter using the received second set of electrical signals in reference to the first set of one or more electrical signals; and presenting, via a display, a visual representation of the determined atrio-esophageal electric coupling parameter.

In some embodiments, the atrio-esophageal electric coupling parameter is expressed as an atrio-esophageal impedance.

In some embodiments, the first electrode is housed in an ablation apparatus and is used in the ablation of tissue in the heart.

In some embodiments, the first electrode is housed in an ablation apparatus, the ablation apparatus having an ablation electrode used in the ablation of tissue in the heart.

In some embodiments, the first set of one or more electrical signals is applied from an electric source (e.g., a current or voltage source) electrically connected to the first electrode, and wherein the first set of one or more electrical signals has an oscillation frequency in a radiofrequency (RF) range (e.g., between 3 kHz and 300 MHz).

In some embodiments, the second electrode comprises a conductive body, wherein the conductive body has a length that spans a portion of the esophagus that substantially overlaps with the heart.

In some embodiments, the conductive body has an effective length (e.g., as a helical shaped structure) that lines the esophageal wall at the region of the esophagus that substantially overlaps with the heart. In some embodiments, the length of the conductive body spans a portion of the esophagus that substantially overlaps with a left atrium of the heart. In some embodiments, the second electrode has a shape and dimension suitable for oral or nasal insertion.

In some embodiments, the method includes measuring, via a third electrode located in the esophagus of the subject, the second set of electrical signals, wherein the second electrode and the third electrode are mounted on a probe body to form an electrode array placed within the esophagus. In some embodiments, the third electrode measures a third set of electrical signals resulting from the applied first set of electrical signals. In some embodiments, the second and third electrodes are each located at a region, of the probe body, that substantially overlaps with the heart (e.g., the left atrium of the heart).

In some embodiments, the method includes receiving, via one or more temperature sensors (e.g., thermistors, RTDs) mounted on the probe body, one or more fourth electrical signals associated with a thermal characteristic of esophageal tissue in contact with the one or more temperature sensors; and in response to the one or more fourth electrical signals, or a derivative parameter derived therefrom (e.g., a temperature value), satisfying a thermal alert condition, triggering the audible or visual alert.

In some embodiments, the method includes introducing, via an irrigation port located on a probe body to which the second electrode is mounted, a cooling solution into the esophagus.

In some embodiments, the method includes drawing, via a suction port located on the probe body, the introduced cooling solution from the esophagus (e.g., into the probe body and out of the esophagus).

In some embodiments, the second electrode is mounted onto a body forming a catheter.

In some embodiments, the method includes receiving an atrium-to-skin impedance parameter measured between a reference electrode located at a location on the skin of the subject and the first electrode; and normalizing a parameter associated with the atrio-esophageal electric coupling with the received atrium-to-skin impedance parameter (e.g., to determine a ratio of Z_{AE}/Z_{AS}, wherein Z_{AE} is the atrio-esophageal electric coupling state expressed in an impedance units, and Z_{AS} is the received atrium-to-skin impedance parameter expressed in impedance units).

In another aspect, a system for preventing esophageal injury during ablation of tissue in the heart is disclosed. The system includes an ablation catheter; an esophageal electrode; and an electric meter (e.g., a LCRZ meter) electrically connected, via a first lead, to the ablation catheter and, via a second lead, to the esophageal electrode, the electric metering having an electric circuit configured to measure an atrio-esophageal electric coupling (e.g., atrio-esophageal impedance) using a measured alternating electric signal captured at the second lead, wherein the measured alternating electric signal results from an applied alternating electric signal generated, by the electric circuit, and applied to the first lead.

In some embodiments, the system includes a three-dimensional (3D) mapping system, the 3D mapping system being coupled to the electric meter to receive i) a first set of data associated with the atrio-esophageal electric coupling and ii) a corresponding second set of data associated with position information collected contemporaneously with the atrio-esophageal electric coupling, the three-dimensional (3D) mapping system being configured to process the first and second set of data to render, via a display, a three-dimensional representation of the atrio-esophageal electric coupling.

In some embodiments, the system includes a vacuum, the vacuum being coupled, via a tube, to a probe body housing the esophageal electrode, the tube terminating at a suction port located at the probe body.

In some embodiments, the system includes a pump coupled, via a second tube, to a probe body housing the esophageal electrode, the second tube terminating at an irrigation port located at the probe body.

In some embodiments, the system includes an imaging system (e.g., ultrasound system, optical coherence tomographic system) coupled, via a cable, to a probe body housing the esophageal electrode, the cable terminating at an imaging probe located at the probe body.

In some embodiments, the system includes a radiofrequency generator, the radiofrequency generator being configured to measure an atrium-to-skin impedance parameter between a reference electrode patch located at a location on the skin of the subject and the ablation catheter, the radiofrequency generator being configured to output the measured atrium-to-skin impedance parameter (e.g., to the electric meter or a computing device operatively connected to the electric meter to receive the atrio-esophageal electric coupling state therefrom) to be used to normalize parameters associated with the measured atrio-esophageal electric coupling.

In another aspect, a method is disclosed for generating a predictive map illustrating levels of risk of esophageal damage due to ablation of tissue in the heart. The method includes receiving, by a processor, a plurality of atrio-esophageal electric coupling data (e.g., an impedance value or an electrical signal associated with atrio-esophageal impedance) from a measurement apparatus, wherein each of the atrio-esophageal electric coupling data includes corresponding spatial position parameters (e.g., x, y, z parameters) to which the data was measured; generating, by the processor, an atrio-esophageal electric coupling map (e.g., an impedance map) using the received atrio-esophageal electric coupling data, wherein the atrio-esophageal electric coupling map comprises a three-dimensional representation of the atrio-esophageal electric coupling; and presenting, by the processor, via a display, the atrio-esophageal electric coupling map.

In some embodiments, the atrio-esophageal electric coupling map has a color scheme that indicates regions of high electric coupling, wherein the regions identify heart tissues having proximity to the esophagus and likely to be associated with esophageal damage if ablative energy is applied there or nearto.

In another aspect, a system for generating a predictive map illustrating levels of risk of esophageal damage due to ablation of tissue in the heart is disclosed. The system includes a processor; and a memory having instructions stored thereon, wherein the instructions, when executed by the processor, cause the processor to: receive a plurality of an atrio-esophageal electric coupling data from a measurement apparatus, wherein each of the atrio-esophageal electric coupling data includes corresponding spatial position parameters (e.g., x, y, z parameters) to which the data was measured; generate an atrio-esophageal electric coupling map (e.g., an impedance map) using the received atrio-esophageal electric coupling data, wherein the atrio-esophageal electric coupling map comprises a three-dimensional representation of the atrio-esophageal electric coupling; and present, via a display, the atrio-esophageal electric coupling map.

In another aspect, an atrio-esophageal probe (apparatus) is disclosed. The atrio-esophageal probe includes a probe body having a connector to releasably connect to one or more cables, the probe body being flexible and having dimensions suitable for oral or nasal insertion into an esophagus of a patient. The atrio-esophageal probe includes a radio-frequency antenna coupled to the probe body, wherein the radio-frequency antenna has a length along the esophagus that spans a region of the esophagus that substantially overlaps with the heart.

In some embodiments, the length along the esophagus is selected from the group consisting of about 5 cm, 6 cm, 7 cm, and 8 cm.

In some embodiments, the length along the esophagus is less than 5 cm.

In some embodiments, the length along the esophagus is greater than 8 cm.

In some embodiments, the radio-frequency antenna has a coil shape. In some embodiments, the radio-frequency antenna has a circular cross-section area.

In some embodiments, the radio-frequency antenna has an impedance of about 50 ohms.

In some embodiments, the radio-frequency antenna comprises a material that is mechanically inert (e.g., does not change or affect placement of the esophagus).

In some embodiments, the radio-frequency antenna comprises a first antenna portion and a second antenna portion.

In some embodiments, the atrio-esophageal probe includes a temperature sensor (e.g., thermistor, RTDs) coupled to the probe body.

In some embodiments, the atrio-esophageal probe includes an irrigation port located in the probe body, the irrigation port coupled to a channel formed within the probe body to terminate at the connector.

In some embodiments, the atrio-esophageal probe includes a suction port located in the probe body, the suction port coupled to a second channel formed within the probe body to terminate at the connector.

### BRIEF DESCRIPTION OF THE DRAWINGS

Embodiments of the present invention may be better understood from the following detailed description when read in conjunction with the accompanying drawings. Such embodiments, which are for illustrative purposes only, depict novel and non-obvious aspects of the invention. The drawings include the following figures:

FIGS. 1A and 1B, respectively, illustrates anatomical relationships between the esophagus and the heart and a schematic of esophageal catheter in position for impedance measurement, in accordance with an illustrative embodiment.

FIGS. 2A and 2B depict diagrams illustrating recordings of atrio-esophageal impedance (Z_{AE}) to assess risk of esophageal injury during cardiac ablation, in accordance with an illustrative embodiment.

FIG. 3 depicts a diagram illustrating recordings of atrio-esophageal impedance (Z_{AE}) using multiple electric meters (e.g., LCRZ meters) to assess risk of esophageal injury during cardiac ablation, in accordance with an illustrative embodiment.

FIG. 4 depicts a diagram of a 3D-mapping system configured to generate a predictive 3D map illustrating levels of risk of esophageal damage due to ablation of tissue in the heart, in accordance with an illustrative embodiment.

FIGS. 5A, 5B, and 5C, each illustrates an example 3D mapping of atrio-esophageal impedance (of a specimen in an animal study), in accordance with an illustrative embodiment.

FIGS. 5D and 5E, each illustrates another example 3D mapping of atrio-esophageal impedance (of another swine), in accordance with an illustrative embodiment.

FIGS. 5F, 5G, 5H, and 5I, each illustrates an example 3D mapping of atrio-esophageal impedance (of a patient), in accordance with an illustrative embodiment.

FIG. 6 depicts a system that monitors esophageal damage by tracking impedances during ablation, in accordance with an illustrative embodiment.

FIGS. 7A and 7B, each depicts photographs of esophageal lesions that had formed due to ablation at a measured low atrio-esophageal impedance (Z_{AE}) site of an animal studied in FIGS. 5A-5C.

FIG. 7C is a photograph taken during an animal validation study used in the mapping of FIGS. 5D and 5E, the photograph shows an esophagus with esophageal lesions that had formed due to ablation at a measured low atrio-esophageal impedance (Z_{AE}) site.

FIG. 7D shows a plot of atrio-esophageal impedance (Z_{AE}) measured at comparative sites of the esophagus when ablation was delivered at corresponding sites in the left atrium.

FIG. 8 depicts a flow chart of a method for preventing esophageal injury during ablation of tissues in the heart, in accordance with an illustrative embodiment.

FIG. 9 depicts a flow chart of a method for generating a predictive map illustrating levels of risk of esophageal damage due to ablation of tissue in the heart.

FIG. 10 depicts a diagram of an apparatus (e.g., an atrio-esophageal probe), in accordance with an illustrative embodiment.

FIG. 11 depicts a diagram of an apparatus (e.g., an atrio-esophageal probe), in accordance with another illustrative embodiment.

FIG. 12 illustrates an exemplary computer that can be used to generate a predictive map illustrating levels of risk of esophageal damage due to ablation of tissue in the heart, in accordance with an illustrative embodiment.

### DETAILED DESCRIPTION OF THE PREFERRED EMBODIMENT

Each and every feature described herein, and each and every combination of two or more of such features, is included within the scope of the present invention provided that the features included in such a combination are not mutually inconsistent.

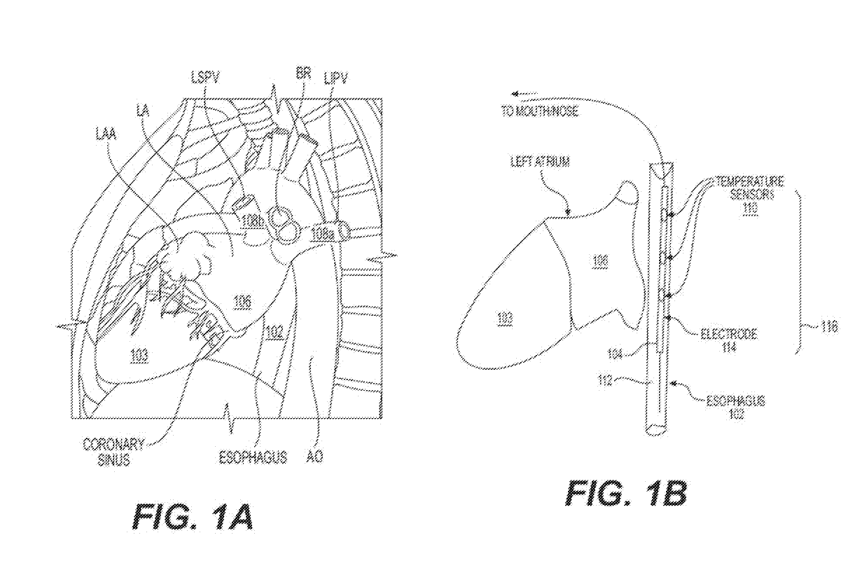

FIGS. 1A and 1B, respectively, illustrates anatomical relationships between the esophagus 102 and the heart 103 and a schematic of an esophageal catheter 104 in position for impedance measurement, in accordance with an illustrative embodiment. As shown in FIG. 1A, the esophagus 102 lies posterior to the left atrium 106 in close proximity to the pulmonary veins (shown as 108a and 108b). As shown in FIG. 1B, which shows a side view of the thoracic area, an esophageal catheter 104 is positioned posterior to the left atrium 106 so that a plurality of temperature sensors 110 located on the catheter 104 may detect esophageal luminal 112 heating and a large electrode 114 located on the catheter 104 covers the entire portion 116, or a substantial portion (e.g., greater than 90%) thereof, of the esophagus that is near the left atrium 106.

In some embodiments, the electrode 114 located on the esophageal catheter 104 spans about 50 percent of the esophagus that is near the left atrium 106. In some embodiments, the electrode 114 located on the esophageal catheter 104 spans about 60 percent of the esophagus that is near the left atrium 106. In some embodiments, the electrode 114 located on the esophageal catheter 104 spans about 70 percent of the esophagus that is near the left atrium 106. In some embodiments, the electrode 114 located on the esophageal catheter 104 spans about 80 percent of the esophagus that is near the left atrium 106. In some embodiments, the electrode 114 located on the esophageal catheter 104 spans about 90 percent of the esophagus that is near the left atrium 106. In some embodiments, the electrode 114 located on the esophageal catheter 104 spans more than the length of the esophagus that is near the left atrium 106.

The esophageal catheter is devised for oral or nasal insertion. The esophageal catheter has a conductive electrode, designed to be long enough to encompass the length of the portion of the esophagus that overlaps with the heart. In some embodiments of the esophageal catheter, an equivalent to a single conductive electrode is implemented with a plurality of electrodes positioned (e.g., in an array or matrix) along the esophageal catheter, the plurality of electrodes being connected (shunted) together to form a single electrical unit. The electrode (single large or multiple shunted smaller ones) of the esophageal catheter is designed so that current density is low to avoid heating of the esophageal lumen, and to cover the length of the esophagus that overlaps with the left atrium. In some embodiments, the esophageal catheter is configured to exhibit high, or higher, impedance for a given set of RF frequencies to be used during the procedure to sufficiently limit current to a desired maximum current density and/or to limit eddy current losses that results from the electrical coupling.

In some embodiments, the esophageal catheter includes a plurality of thermistors, each configured to sample esophageal luminal temperature along the esophagus. It is contemplated that other types of temperature sensing mechanisms can be used. The thermistors (or temperature sensing mechanisms), in some embodiments, are used to trigger a cutoff switch (e.g. hard wired or soft wired in, or to, the power source of the ablation system) that stops the ablation. In some embodiments, the switch is triggered once the thermistor or temperature sensing mechanisms is subjected to a pre-defined temperature (e.g., a maximum temperature limit). In conjunction with, or as an alternative to the maximum temperature limit, the switch, in some embodiments, is configured to trigger once the thermistor or temperature sensing mechanisms is subjected to a pre-defined change in temperature (e.g., a maximum AT limit).

In some embodiments, the esophageal catheter includes one or more irrigation ports to provide esophageal cooling in case of excessive heating. The irrigation port is connected to channel within the esophageal catheter and terminates at a connector that is connected to a pump. The irrigation port allows cooling solutions to be infused into the esophageal lumen. In some embodiments, the irrigation port includes one or more openings to introduce fluids at either the same general area or to different areas. The irrigation port may be located proximal or distal to the esophageal electrode. In some embodiments, thermistors (or temperature sensing mechanisms) (same or separate from those used for temperature cutoff) are used to control flow of the cooling solutions to the irrigation port (e.g., by varying output of the pump or adjusting position of flow valves located in the irrigation circuit).

In some embodiments, the esophageal catheter includes a suction port. The suction port is coupled to a channel formed within the probe body to terminate at the connector.

In some embodiments, the esophageal catheter includes an imaging port. The catheter may have a lumen to incorporate probes such as rotational ultrasound or optical coherence tomography to provide measurements from which an image can be derived.

In some embodiments, once inserted via the mouth or the nose into the esophagus, the catheter is positioned so that the temperature sensors sample the portion of the esophagus in contact with the left atrium as guided by fluoroscopy.

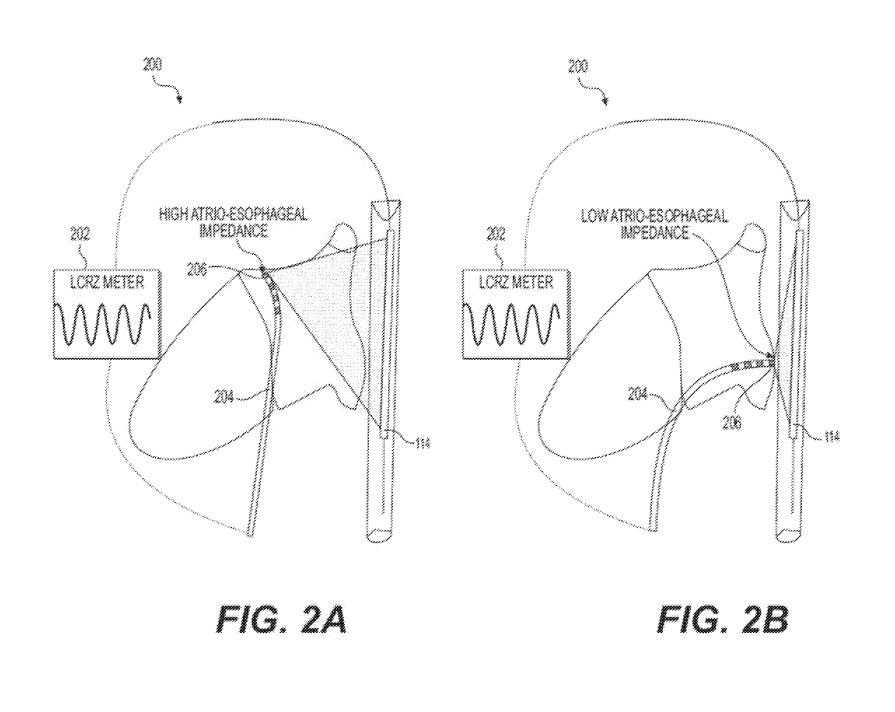

FIGS. 2A and 2B, each depicts diagrams illustrating a system 200 for recording trio-esophageal impedance (Z_{AE}), in accordance with an illustrative embodiment.

As shown in FIGS. 2A and 2B, an electric meter 202 (for example, an “LCRZ meter” 202, where “L” refers to inductance, “C” refers to capacitance, “R” refers to resistance, and “Z” refers to impedance) is used for atrio-esophageal impedance (Z_{AE}) measurement. Electric meter 202 can include any instrument or data acquisition system configurable to measure impedance, including hand held instruments, dedicated laboratory equipment, or customizable data acquisition systems. The LCRZ meter 202, in this example, is configured to generate an alternating current between the esophageal electrode 114 of the esophageal catheter 104 and an ablation catheter 204 by injecting alternating current (e.g., RF current) to an electrode of the ablation catheter 204. The resulting electric coupling (e.g., observed or measured waveform of the LCRZ meter) between the esophageal electrode 114 and the ablation catheter 204 is used to measure, or characterize, impedance (Z) (also referred to herein as “atrio-esophageal impedance (Z_{AE})”) between the esophageal electrode 114 and the ablation catheter 206 (e.g., the tip of the catheter). Impedance (Z) is generally defined as the total opposition a tissue and electrode circuit offers to the flow of an alternating current (AC) at a given frequency, and is represented as a complex quantity. When the catheter (e.g., 204), specifically the tip thereof, is manipulated in the left atrium, the atrio-esophageal impedance (Z_{AE}) can be measured. Atrio-esophageal impedance (Z_{AE}) is a measure of “electrical coupling” between the esophagus electrode and the ablation catheter tip and varies depending on the distance between the two.

Referring still to FIGS. 2A and 2B, an alternating current (shown as a shaded triangle) is passed from the esophagus to the ablation catheter tip. In FIG. 2A, at sites in the heart that is far from the esophagus, Z_{AE} is high. In FIG. 2B, at sites in the heart that is close to the esophagus, Z_{AE} is low. It is contemplated that the LCRZ meter 202 can apply the alternating current to the esophageal electrode 114 or to the ablation catheter 204. That is, the AC output lead of the LCRZ meter 202 can be coupled to the esophageal electrode 114 and the return lead of the LCRZ meter 202 coupled to the ablation catheter 204, or the AC output lead can be coupled to the ablation catheter 204 and the return lead coupled to the esophageal electrode 114.

Multiple electrodes in the ablation catheter (as opposed to a single distal one located at, or near, the tip) can be used as well.

Normalizing Measured Atrio-Esophageal Impedance

In another aspect, a system and method is disclosed to normalize the measured atrio-esophageal impedance to account for non-atrio-esophageal coupling determinants. In some embodiments, the system is configured to take a second measurement of unipolar impedance (skin reference electrode) and use that measurement to normalize the measured atrio-esophageal impedance.

Radiofrequency (RF) generators can be used to pass radiofrequency energy between a skin reference electrode patch and an electrode in the ablation catheter tip. These generators measure impedance of this circuit to assess the tissue properties of cardiac tissue in contact with the ablation catheter tip electrode. When used in the atrium, this impedance is referred as “atrium-to-skin” impedance (Z_{AS}). Atrium-to-skin impedance (Z_{AS}) can vary based on the tissue characteristics in contact with the electrode, as well as the chamber size and the pool of blood surrounding the ablation catheter tip electrode. Atrium-to-skin impedance (Z_{AS}) is routinely measured during catheter ablation procedures.

In some embodiments, atrium-to-skin impedance (Z_{AS}) is measured, via a single RF generator, connected in parallel with an atrio-esophageal impedance (Z_{AE}) measurement. The measurements of atrium-to-skin impedance (Z_{AS}) and atrio-esophageal impedance (Z_{AE}) may be performed, for all locations of the heart, as the catheter is moved in the atrial chamber, to normalize the measured atrio-esophageal impedance (Z_{AE}). In some embodiments, the measured atrio-esophageal impedance (Z_{AE}) is normalized as a ratio of

$$\frac{{Z}_{AE}}{{Z}_{AS}}$$

to correct for impedance changes caused by non-atrio-esophageal coupling determinants.

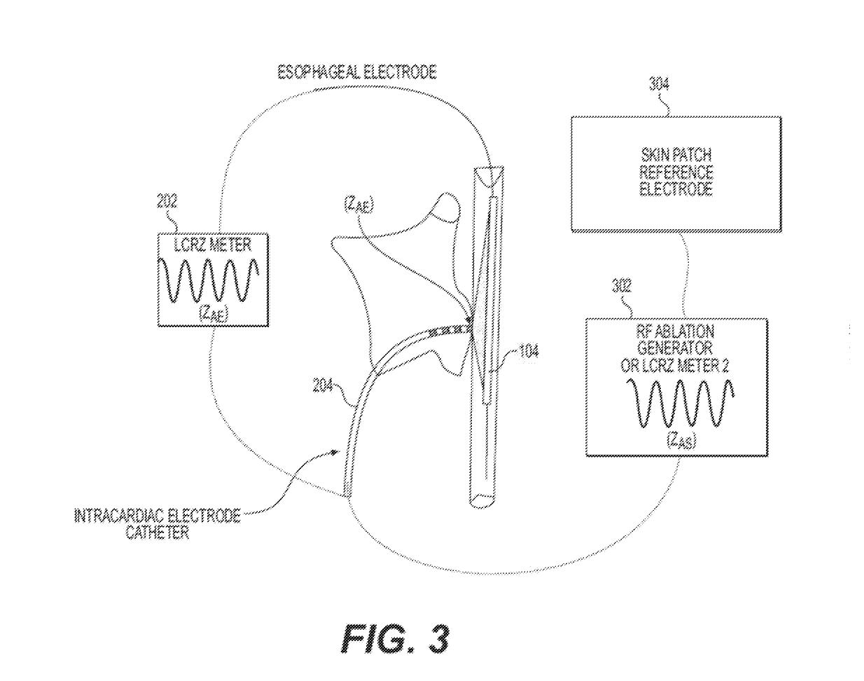

In some embodiments, a second LCRZ meter is used to independently measure and calculate Z_{AS} as the catheter is moved, or roved, around the atrium (rather than using a RF generator). FIG. 3 depicts a diagram illustrating recording of atrio-esophageal impedance (Z_{AE}) using a second LCRZ meter (shown as “LCRZ meter 2”), in accordance with an illustrative embodiment. As shown in FIG. 3, a first LCRZ meter 202 (shown as “LCRZ meter” 202) is used to measure atrio-esophageal impedance (Z_{AE}), and a second LCRZ meter 302 (shown as “RF ablation generator or LCRZ meter 2” 302) is used to measure atrium-to-skin impedance (Z_{AS}). Specifically, the first LCRZ meter 202 is coupled to the esophageal electrode 114 of the esophageal catheter 104 and the electrode of the ablation catheter 204 and is configured to generate a RF coupling between the esophageal electrode 114 of the esophageal catheter 104 and the electrode of the ablation catheter 204. The second LCRZ meter 302, in some embodiments, is coupled to the same ablation catheter 204 and a skin patch reference electrode 304 and is configured to generate a second RF coupling between the ablation catheter 204 and the skin patch reference electrode 304 via injection of an alternating current to an electrode of the ablation catheter 204 or the skin patch reference electrode 304.

Visualization of 3D Mapping of Atrio-Esophageal Impedance

In another aspect, visualization of 3D mapping of atrio-esophageal impedance, in areas in the heart, may be performed, during ablation procedures, to indicate and/or highlight areas of risk, particularly, areas of high risk. The 3D mapping may be performed by a system having the measurement devices, described above, integrated with a three-dimensional mapping system.

The 3D mapping system acquires three-dimensional coordinates (e.g., x, y, and z coordinates) of the ablation catheter tip electrode as the catheter is moved, or roved, around the left atrium. Such coordinates may be obtained by standard commercial mapping systems such as an impedance-based mapping or magnetic-localization system. Example of an impedance-based mapping system is the EnSite™ NavX™ system developed by St. Jude Medical (St. Paul, Minn.).

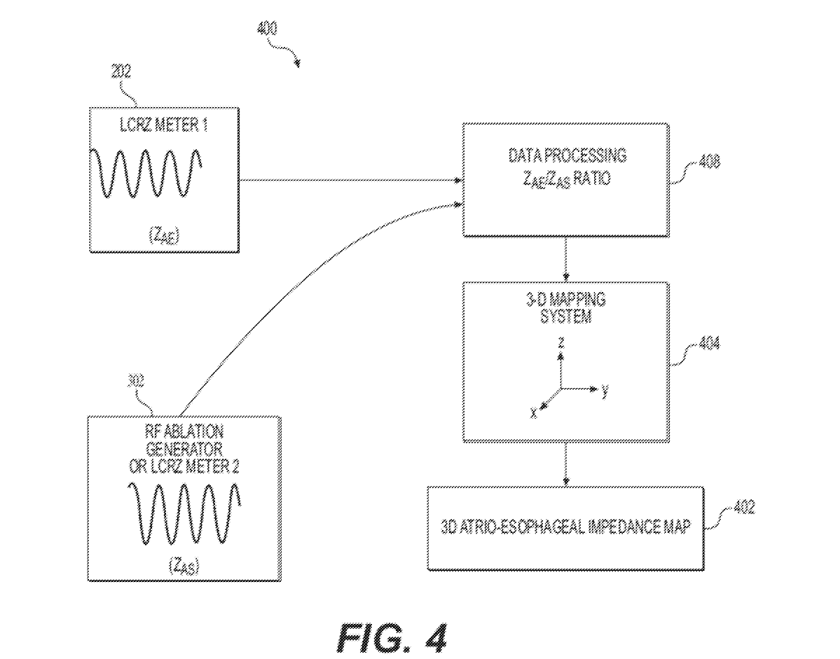

FIG. 4 depicts a diagram of a system 400 configured to generate a 3D atrio-esophageal impedance map 402, e.g., as a predictive 3D map, that illustrates levels of risk of esophageal damage due to ablation of tissue in the heart, in accordance with an illustrative embodiment. As shown in FIG. 4, the system 400 includes a first LCRZ meter 202 (shown as “LCRZ meter 1” 202) and a RF ablation generator or second LCRZ meter 406 (shown as “RF ablation generator or LCRZ meter 2” 302), each operatively coupled to a 3-D mapping system 404. The first LCRZ meter 202 is configured to measure and record atrio-esophageal impedance (Z_{AE}), as for example described in relation to FIGS. 2A, 2B, and 3, and the RF ablation generator or second LCRZ meter 302 is configured to measure and record atrium-to-skin unipolar impedance (Z_{AS}), as for example described in relation to FIG. 3, as the catheter is moved around the atrium. The atrium-to-skin impedance (Z_{AS}) data and atrio-esophageal impedance (Z_{AE}) data are combined, e.g., via a computer 408 (shown as “data processing” 408), for normalization and other analysis, and the resulting data are then fed to the 3D-mapping system 404 (which may reside on the same or different computer). The 3-D mapping system 404 combines the normalized atrio-esophageal impedance (Z_{AE}) data with the collected coordinate locations of the roving catheter (e.g., gathered by the 3-D mapping system) and creates a map 402 of normalized atrio-esophageal impedance (Z_{AE}) (shown as “3D Atrio-esophageal impedance map).

In some embodiments, the 3-D mapping system 404 combines the un-normalized atrio-esophageal impedance (Z_{AE}) with the collected coordinate locations to create a map of measured atrio-esophageal impedance (Z_{AE}).

The 3D atrio-esophageal impedance map 402 may be used in many ways. The 3D atrio-esophageal impedance map 402 may be presented for presentation on a display (e.g., same or different computer). In some embodiments, the 3D atrio-esophageal impedance map 402 is presented in a report (digital or hardcopy).

Predict Areas of High Risk of Esophageal Damage

In an embodiment, the 3D atrio-esophageal impedance map 402 is used to predict areas of high risk of esophageal damage according to a first mode of operation (e.g., during the planning of an ablation procedure). The system 400, as described in relation to FIG. 4, records atrio-esophageal impedance (Z_{AE}) using any of the electrodes that are part of a roving catheter (e.g., 204) inserted in the heart (e.g., ablation or diagnostic catheters) and using the esophageal electrode (e.g., 114) as a reference. As the catheter (e.g., 204) roves within the chamber of the heart, the local atrio-esophageal impedance (Z_{AE}) at each position is measured by the system. Using the 3D mapping system (e.g., 404) (e.g., a commercial 3D mapping system), the value of the atrio-esophageal impedance (Z_{AE}) is combined with the collected position (x, y, and z) of the catheter (e.g., 204) concurrently sampled and collected at the time of measurement to form the 3D atrio-esophageal impedance map. The atrio-esophageal impedance (Z_{AE}) can then be color-coded and added to the 3D geometry of the left atrium or any chamber being mapped.

Once the map (e.g., 402) is generated, the operator can use the map (e.g., 402) to readily observe i) areas (e.g., identified areas) of low atrio-esophageal impedance (Z_{AE}) and/or risk, which are characterized as a region physically closest to the esophagus and ii) areas (e.g., identified areas) of the highest risk of suffering from esophageal damage if ablative energy is applied there. Without being tied to a theory, the critical characteristic of atrio-esophageal impedance (Z_{AE}) is that, although it correlates with physical distance, it is a direct measurement of electrical coupling between the catheter (e.g., 204) roving in the atrium and the electrode (e.g., 114) located in the esophagus. Since such electrical coupling is a direct parameter influencing transmission of radiofrequency energy through tissues, it is uniquely-well suited to predict esophageal damage when delivering radiofrequency to atrial tissues.

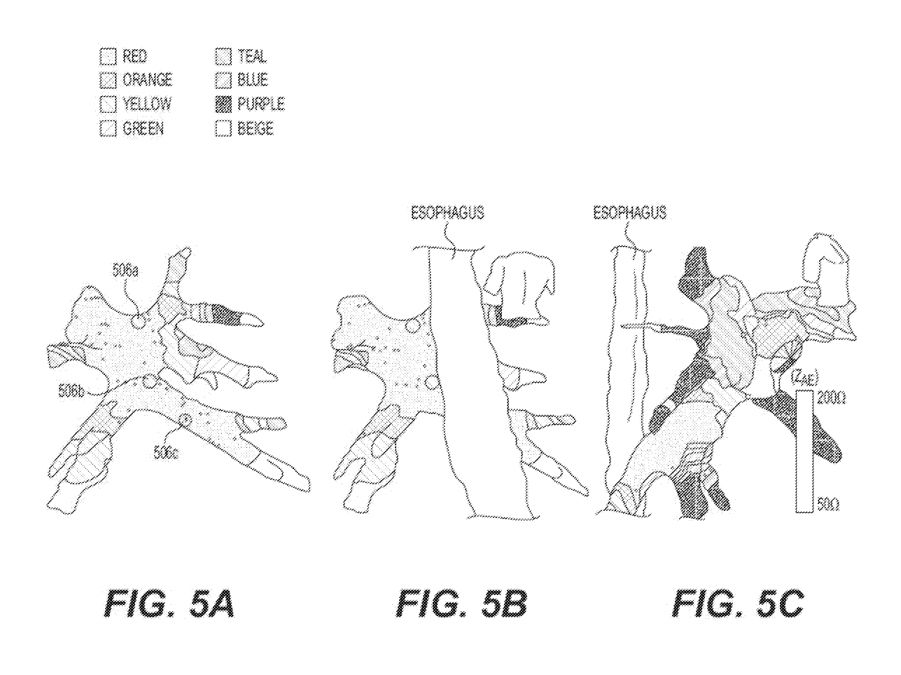

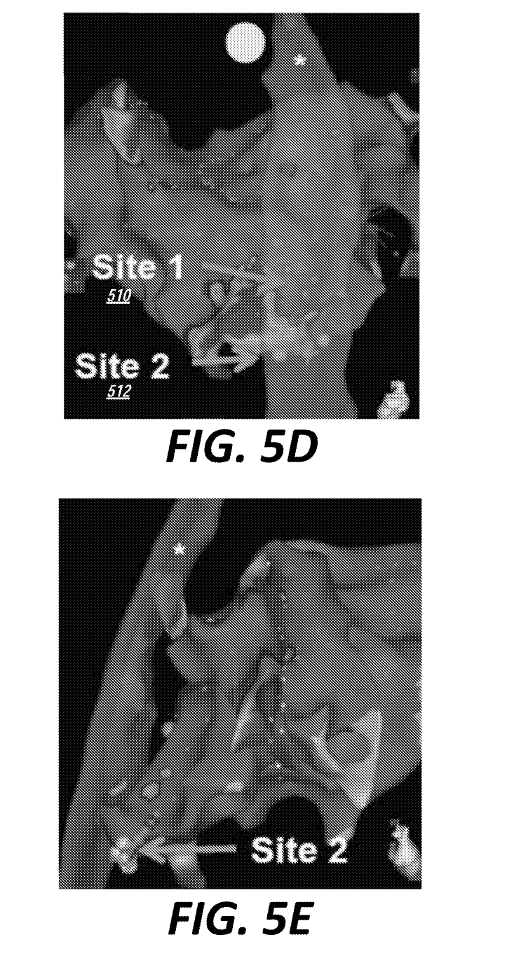

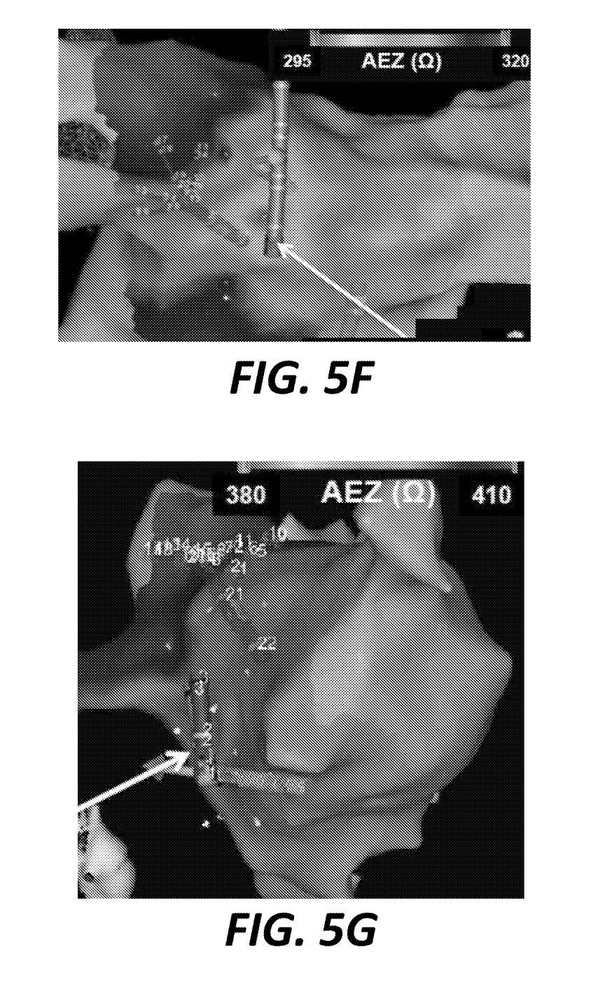

FIGS. 5A, 5B, and 5C, each illustrates an example 3D mapping of atrio-esophageal impedance (Z_{AE}) on the left atrial 3-dimensional geometry (of a specimen in an animal study), in accordance with an illustrative embodiment. Specifically, FIG. 5A illustrates 3D mapping of atrio-esophageal impedance (Z_{AE}) in a swine left atrium. The figure is coded (in various shades of color) to correspond to atrio-esophageal impedance (Z_{AE}) values. As shown in FIG. 5A, the lowest atrio-esophageal impedance (Z_{AE}) (marked by the symbol “*”, shown as 502) appears to occur in the right inferior pulmonary vein.

FIG. 5B illustrates the 3D map obtained in FIG. 5A with the esophageal geometry superimposed. Each of FIGS. 5A and 5B shows a posterior view. FIG. 5C shows a right lateral view of a second 3D mapping of atrio-esophageal impedance (Z_{AE}) of a second swine. In FIG. 5C, the lowest atrio-esophageal impedance (Z_{AE}) appears to be close to the esophagus (shown as 504).

Monitoring Esophageal Damage During Ablation

In another embodiment, the 3D atrio-esophageal impedance map 402 is used to detect the occurrence of esophageal damage according to a second mode of operation (e.g., to monitor for damage during ablation). In this monitoring mode, atrio-esophageal impedance (Z_{AE}) is monitored during ablation using, in some embodiments, an ablating or a non-ablating electrode of the ablation catheter and the esophageal electrode as a reference. Radiofrequency ablation catheters may have a plurality of electrodes besides the distal ablation electrode. During radiofrequency ablation, energy is passed between the distal ablation electrode and a patch in the skin. As opposed to the Z_{AE} mapping mode—in which the distal electrode is used to measure Z_{AE} during ablation—the distal electrode may be subject to impedance changes that are a reflection of changes in the myocardial tissue being ablated. Without being tied to a theory, it is believed that when using the distal ablating electrode to measure atrio-esophageal impedance (Z_{AE}), changes in atrio-esophageal impedance (Z_{AE}) during ablation are a function of changes in myocardial impedance due to ablation, as well as changes in esophagus tissue integrity. Thus, another non-ablating electrode within the ablation catheter may be used to measure atrio-esophageal impedance (Z_{AE}) during ablation.

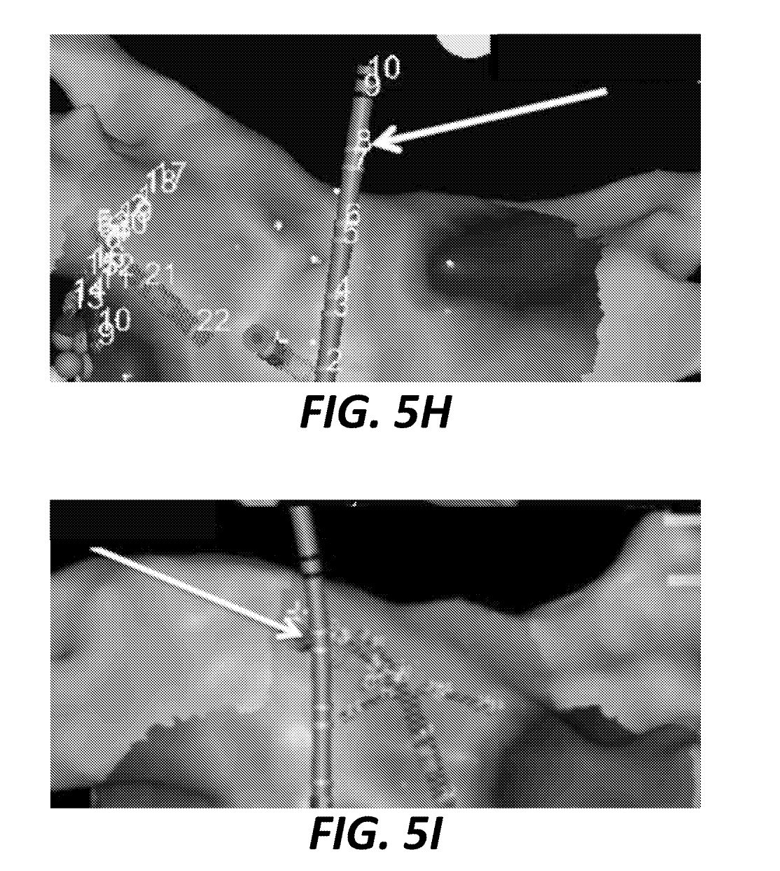

FIG. 6 depicts a system 600 that monitors esophageal damage by tracking impedances during ablation, in accordance with an illustrative embodiment. The system 600 tracks real time impedances using several catheter configurations.

ΔZ_{AE1}: tracked atrio-esophageal impedance, or changes thereof, between a distal (ablative) electrode 602 located on the ablation catheter 204 and the electrodes 114 of the esophageal catheter 104.

ΔZ_{AE2}: tracked atrio-esophageal impedance, or changes thereof, between a proximal (non-ablative) electrode 604 located on the ablation catheter 204 and the electrodes 114 of the esophageal catheter 104, which will not reflect changes created by ablated myocardium.

ΔZ_{AS}: tracked unipolar impedance, or changes thereof, between the distal (ablative) electrode 602 and the skin (e.g., via a skin patch reference electrode 606) and reflects myocardial tissue damage.

Because, measuring changes in atrio-esophageal impedance (ΔZ_{AE}) to track changes in tissue created by ablation may mostly reflect physiologic changes in the myocardial tissue being ablated and not reflect physiologic changes in the esophagus, to measure the latter, the system 600 uses other electrodes present in the ablation catheter 204 to track atrio-esophageal impedance (Z_{AE2}) alone or in combination with the distal electrode 602 (which can be used to track atrio-esophageal impedance, Z_{AE1}). In some embodiments, in the atrio-esophageal impedance (Z_{AE2}) monitoring mode, the proximal electrode 604 is additionally used to directly pass a second (e.g., low-grade) alternating current to the electrodes 114 of esophageal catheter 104 located in the esophagus to measure atrio-esophageal tissue impedance (Z_{AE2}) of the esophagus 102. In some embodiments, the second alternating current has a lower power output and/or a different frequency, or set of frequencies, than the alternating current injected to the distal electrode 602 (e.g., to measure atrio-esophageal tissue impedance, Z_{AE1}). During ablation, changes in the tissue integrity between esophagus tissue 102 and the atrium 106 can be detected by changes in atrio-esophageal impedance (ΔZ_{AE}). Using the proximal electrode 604, atrio-esophageal impedance (Z_{AE2}) is less likely to reflect changes in local myocardial tissue being ablated.

Additionally, the contribution of myocardial tissue ablation to impedance changes can be effectively tracked by measuring impedance (i.e., atrium-to-skin impedance (Z_{AS})) from the ablating electrode 602 of the ablation catheter 204 to the reference skin electrode patch 606 (shown as “skin patch reference electrode” 606). Using atrium-to-skin impedance (Z_{AS}) as a reference, changes in atrio-esophageal impedance (e.g., ΔZ_{AE1} or ΔZ_{AE2}) that goes beyond those of atrium-to-skin impedance (Z_{AS}) are reflective, in some embodiments, of nonmyocardial tissue damage and potential of esophageal lesion creation. That is, by tracking several impedances at once: i) atrio-esophageal impedance (Z_{AE1}) from the distal ablative electrode 602 to esophagus 102, ii) atrio-esophageal impedance (Z_{AE2}) from the proximal electrode 604 to esophagus 102, iii) and atrium-to-skin impedance (Z_{AS}) from the distal ablative electrode 602 of the ablation catheter 204 to the skin patch electrode 606, the system 600 can discriminate changes in atrio-esophageal impedance (ΔZ_{AE1} and/or ΔZ_{AE2}) that are not due to myocardial tissue ablation. Normalizing changes in atrio-esophageal impedance (ΔZ_{AE1} and/or ΔZ_{AE2}) to changes in atrium-to-skin impedance (Z_{AS})—or subtracting them—will eliminate changes from myocardial ablation.

Experimental Results—Animal Validation Study

The concept has been validated in 10 swine. Using a large surface electrode in the esophagus and a roving ablation catheter in the left atrium, atrio-esophageal impedance (Z_{AE}) is measured in different areas of the left atrium. The local atrio-esophageal impedance (Z_{AE}) of different sites was calculated and added to the 3-dimensional geometry of the left atrium, to create a 3D atrio-esophageal impedance (Z_{AE}) map (as for example shown in FIGS. 5A-5C). It was observed that areas that were closest to the esophagus had the lowest atrio-esophageal impedance (Z_{AE}) values.

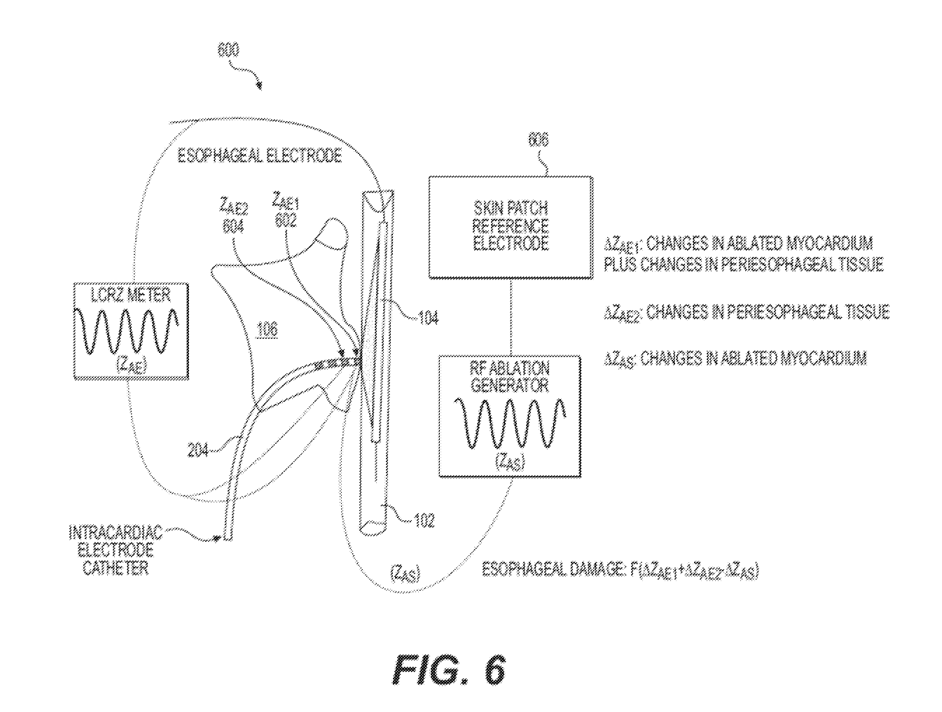

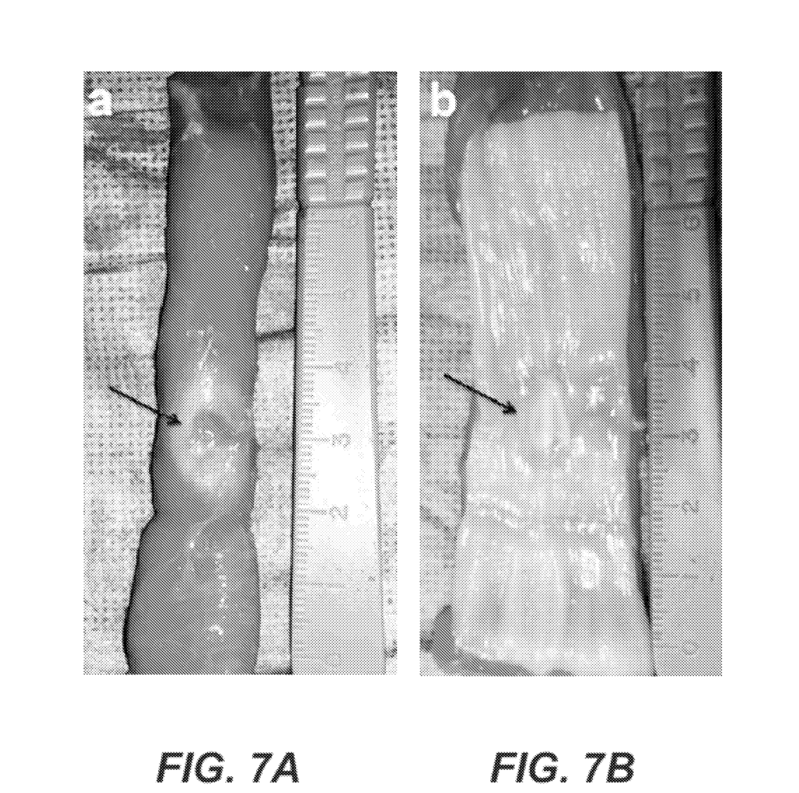

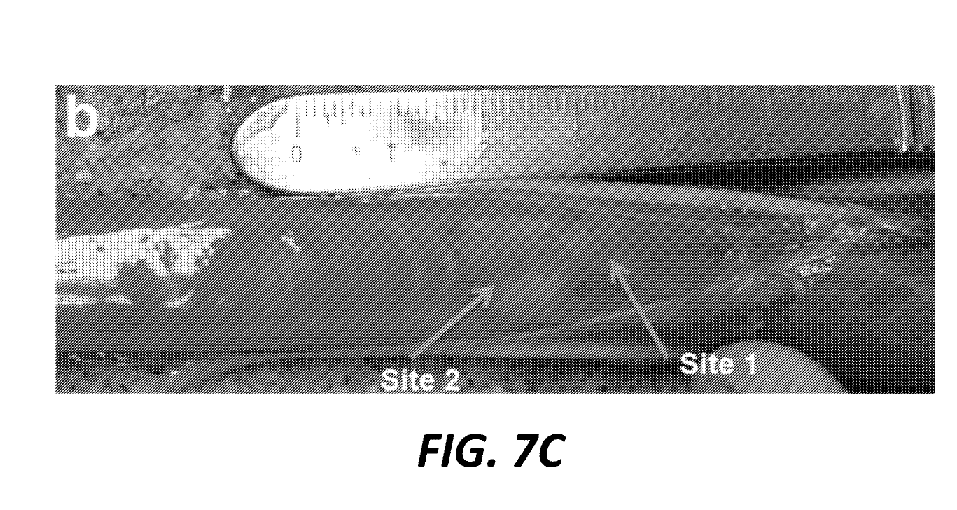

A key validation issue is that radiofrequency application (i.e., ablation) in areas of low atrio-esophageal impedance (Z_{AE}) correlates or leads to esophageal damage, whereas ablation in areas of “normal” impedance does not damage the esophagus. Radiofrequency applications (i.e., ablation) were performed at 3 sites of the left atrium. The 3 sites are shown in yellow dots in FIG. 5A (shown as 506a, 506b, and 506c). Only the lower site (506c)—where Z_{AE} was lowest (502)—led to ablation-induced damage to the adventitia of the esophagus. FIGS. 7A and 7B depict diagrams of esophageal lesions that had occurred after ablation at the low Z_{AE} site (502). As shown in FIGS. 7A and 7B, esophageal lesions occur after ablation only at the low Z_{AE} site. Of the 3 lesions (yellow dots in FIG. 5A) performed in the posterior left atrium, only the one at a low Z_{AE} site led to esophageal damage. FIGS. 7A and 7B, each depicts a photograph of esophageal lesions that had formed due to ablation at a measured low atrio-esophageal impedance (Z_{AE}) site of an animal studied in FIGS. 5A-5C. Specifically, FIG. 7A shows an explanted esophagus with obvious lesions in the adventitia, and FIG. 7B shows the explanted damaged esophagus of FIG. 7A bisected to further show lesions in the submucosa.

Experimental Results—Integrity Assessment of Esophageal Tissue Via Atrio-Esophageal Impedance (Z_{AE}) Measurements

FIGS. 5D and 5E, each illustrates the 3D mapping of atrio-esophageal impedance in an animal study to validate atrio-esophageal impedance (Z_{AE}) uses in assessing esophageal tissue, in accordance with an illustrative embodiment. FIG. 7C is a photograph taken during another animal validation study to determine atrio-esophageal impedance (Z_{AE}) uses in assessing esophageal tissue, in accordance with another embodiment.

It is contemplated that there is a clinical need is to detect the onset of esophageal damage during ablation to avoid and prevent inadvertent damage to the esophagus. It is also contemplated that there is a clinical need to ablate safely, and prevent or minimize esophageal damage, when deliberately performing ablation at areas of short atrio-esophageal distance (AED), e.g., by modulating ablation parameters (duration, power, contact force, etc.) may be modulated as needed. For example, by knowing the precise time that the onset of esophageal tissue injury is occurring, ablation can be stopped to minimize injury and prevent permanent damage, or ablation parameters can be adjusted accordingly.

It is shown in a study that, during ablation, atrio-esophageal impedance (Z_{AE}) variations can also be monitored to track the integrity of esophageal tissue. That is, changes in tissue integrity during ablation would affect the electrical conductive properties so as to be reflected by changes in atrio-esophageal impedance (ΔZ_{AE}). During the study, radiofrequency ablation was performed in the left atrium in in-vivo swine. The swine left atrium has a large inferior vein that comes in direct apposition to the esophagus. In each pig, two sites were tested in the left atrium in which radiofrequency energy was delivering at each site for 2 minutes at 35 W (Watts). Ablation sites were chosen to be in close proximity to the esophagus, as measured by 3-dimensional maps, but one site was in direct contact with the esophagus while the other was not. During the ablation, continuous atrio-esophageal impedance (Z_{AE}) monitoring was performed, the 3D mapping of acquired atrio-esophageal impedance generated from a study is shown in FIGS. 5D and 5E. Radiofrequency application was delivered with equal parameters (i.e., 35 Watts for 120 seconds). As shown in FIGS. 5D and 5E, a left atrial map is presented with ablation sites 1 and 2 indicated (shown as “Site 1” 510 and “Site 2” 512).

It is found that, at site 1, the atrio-esophageal impedance (Z_{AE}) was observed to remain stable, and there were no esophageal lesions formed. At site 2, it is observed that the change in atrio-esophageal impedance (ΔZ_{AE}) increased, in some instances, by more than 500 percent (%) and the changes in atrio-esophageal impedance (ΔZ_{AE}) appears to correlate with injuries to the esophageal wall. FIG. 7D shows a plot of the change in atrio-esophageal impedance (ΔZ_{AE}) measured at comparative sites of the esophagus when ablation was delivered at corresponding sites in the left atrium. As shown in FIG. 7D, it is observed that the change in atrio-esophageal impedance (ΔZ_{AE}) increased to substantially elevated levels in three instances (shown as 702, 704, and 706).

To this end, radiofrequency application in the site separated from the esophagus showed minor Z_{AE} variations and did not lead to visible esophageal damage on autopsy examination. Whereas, radiofrequency application in the site closest to the esophagus led to profound rises in changes in atrio-esophageal impedance (ΔZ_{AE}), as high as 500% increases from baseline. On necropsy, an obvious lesion in the esophageal wall was found. Only those radiofrequency applications resulting in esophageal damage had this impedance rises. Thus, the concept of AEZ tracking to detect esophageal lesions is validated.

Experimental Results—Human Validation Study

Validation of the use of atrio-esophageal impedance (Z_{AE}) to predict risk of esophageal thermal injury has been validated, on at least 12 patients, by performed left atrial regional maps of atrio-esophageal impedance (Z_{AE}) and assessing the correlation between atrio-esophageal distance and rises in luminal esophageal temperature.

In each study, a decapolar mapping catheter (i.e., the DecaNav® manufactured by Biosense Webster, Inc. of South Diamond Bar, CA) was inserted in the esophagus of a given patient along with a LET (Luminal esophageal temperature) thermistor instrument configured with 10 electrodes placed intermittently along the surface of thermistor instrument. The 10 electrodes were connected and shunted together outside the body; the shunt combines the multiple electrodes into a single, combined large surface esophageal reference electrode. The shunted poles of each electrodes were terminated into a single connector that is plugged into a reference patch port of a radiofrequency generator (i.e., the SmartAblate™ system manufactured by Stockert GmbH, Germany).

With the above discussed electrode configuration disposed on the thermistor, the radiofrequency generator continuously reported the atrio-esophageal impedance (Z_{AE}) of the tissue and cavity space between the reference electrode and the ablation catheter. The measured atrio-esophageal impedance (Z_{AE}) outputs of the radiofrequency generator are continuously outputted to a 3-dimensional (3D) mapping system. The THERMOCOOL® SMARTTOUCH™ (manufactured by Biosense Webster, Inc.) was used in the study.

The three-dimensional (3D) mapping system generates a regional atrio-esophageal impedance (Z_{AE}) maps from the measured atrio-esophageal impedance (Z_{AE}) by combining the impedance data with 3D coordinate data (e.g., the atrio-esophageal distance, AED) of the ablation catheter localized by the three-dimensional (3D) mapping system. The atrio-esophageal distance (AED) was measured, for use in the 3D maps, as measurements of the distance between the esophageal electrode and points in the left atrium (LA).

The study was performed in 12 patients aged 46-79 years (mean 70±9 years; 58% male; 50% persistent AF), undergoing catheter ablation of atrial fibrillation (AF). End-expiratory, diastolic atrio-esophageal impedance (Z_{AE}) maps of the left atrium were successfully constructed by color-coding point-by-point atrio-esophageal impedance (Z_{AE}). All patients had lowest atrio-esophageal impedance (Z_{AE}) at points of shortest atrio-esophageal distance. The results verified the consistent correlation between the region of maximum temperature rise (mean 0.9° C.±0.7° C.), the shortest atrio-esophageal distance, and the region of lowest atrio-esophageal impedance (Z_{AE}).

FIGS. 5F, 5G, 5H, and 5I, each illustrates an example 3D mapping of atrio-esophageal impedance of the left atrial map (of a patient), in accordance with an illustrative embodiment. Each view of the 3D mapping shows a lowest atrio-esophageal impedance (Z_{AE}) reading at the site (shown as 508a, 508b, 508c, and 508d) closest to the esophagus. The maps show lowest atrio-esophageal impedance (Z_{AE}) at the sites closest to the esophagus in red.

Method of Operation

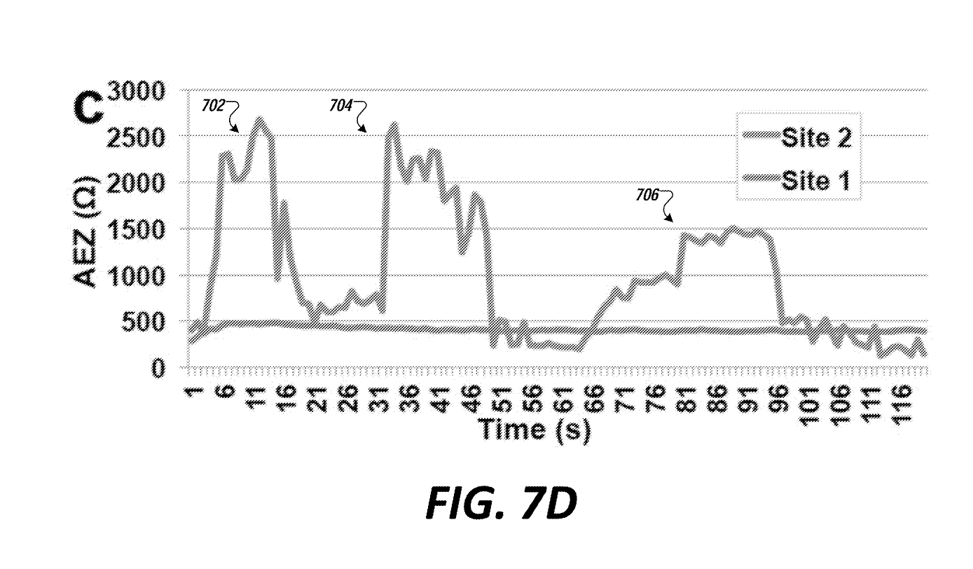

FIG. 8 depicts a flow chart of a method 800 of preventing esophageal injury during ablation of tissues in the heart, in accordance with an illustrative embodiment. The method 800 includes applying, to a first electrode (e.g., ablation catheter 204) located within a chamber (e.g., the atrium) of the heart of a subject (e.g., a person, an animal, etc.), a first set of one or more electrical signals (e.g., via a current source, wherein the one or more signals have RF frequencies) (step 802). The RF frequencies may be in the radiofrequency (RF) range (e.g., between 3 kHz and 300 MHz).

The method 800 includes measuring, from a second electrode (e.g., esophageal electrode 104) located in the esophagus of the subject, a second set of electrical signals resulting from the first set of one or more electrical signals being applied to the first electrode, wherein the second set of electrical signals characterizes an atrio-esophageal electric coupling (e.g., an atrio-esophageal impedance) between the first electrode located in the chamber of the heart and the second electrode located in the esophagus (step 804).

The method 800 includes, in response to the atrio-esophageal electric coupling, or a derivative parameter derived therefrom (e.g., proximity value), satisfying an alert condition (e.g., an impedance or derived proximity value being within an alert condition, or outside a non-alert condition, e.g., based on analog or numerically-derived threshold), causing an audible or visual alert to be generated (step 806).

In some embodiments, the alert condition may be based on the measured second set of electrical signals (e.g., an analog signal or a digital signal), an analog-to-digital conversion value associated with the conversion of the second set of electrical signals, a parameter such as an impedance value derived from the A/D conversion of the second set of electrical signals, or a derived parameter (such as proximity or risk) derived from the impedance value. The alert condition may include a threshold value or an operational range that defines an acceptable or unacceptable state for each of the type of above-discussed basis used in the comparison.

In some embodiments, the audible alert is generated from an analog or digital circuit that drives a speaker based on, e.g., an analog or digital comparator circuit. In other embodiments, the audible alert is generated by a speaker triggered, by a computing device, to output an audio file.

The visual alert, in some embodiments, may be generated from an analog circuit or digital circuit that drives one or more light-emitting-diodes, lamps, monitors, and the like. In some embodiments, the visual alert is a visual representation of a rendered image of 3D atrio-esophageal impedance map or risk map derived therefrom.

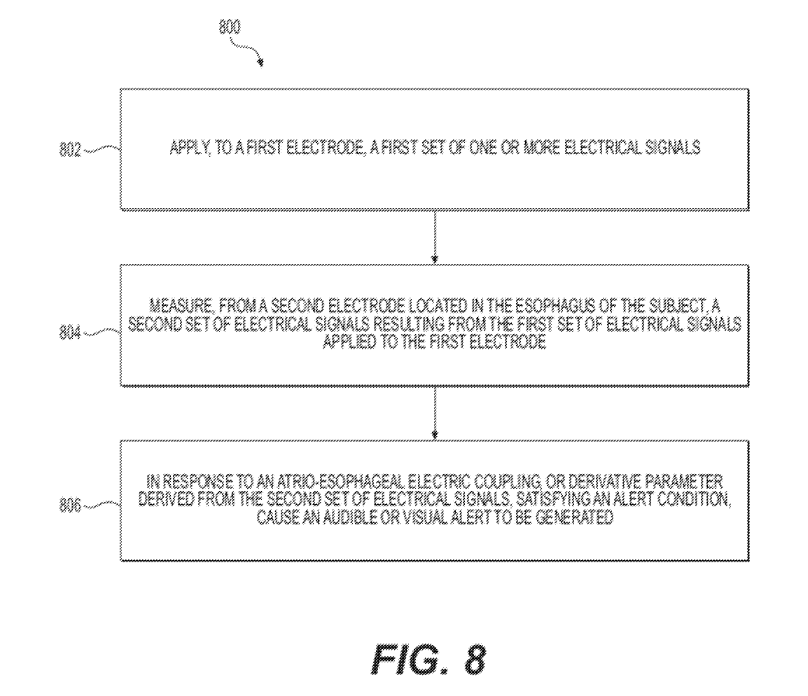

FIG. 9 depicts a flow chart of a method 900 for generating a predictive map illustrating levels of risk of esophageal damage due to ablation of tissue in the heart. The method 900 includes receiving, by a processor, a plurality of atrio-esophageal electric coupling data (e.g., an impedance value, a signal associated with atrio-esophageal impedance) received from a measurement apparatus (e.g., a LCRZ meter), wherein each of the atrio-esophageal electric coupling data includes corresponding spatial position parameters (e.g., x, y, z parameters) to which the data was measured (e.g., via a 3D mapping system such that those described in relation to FIG. 4) (step 902).

The method 900 includes generating, by the processor, an atrio-esophageal electric coupling map (e.g., a 3D atrio-esophageal impedance map) using the received atrio-esophageal electric coupling data, wherein the atrio-esophageal electric coupling map comprises a three-dimensional representation of the atrio-esophageal electric coupling (step 904).

The method 900 includes presenting, by the processor, via a display, the atrio-esophageal electric coupling map (step 906).

Example Atrio-Esophageal Probe

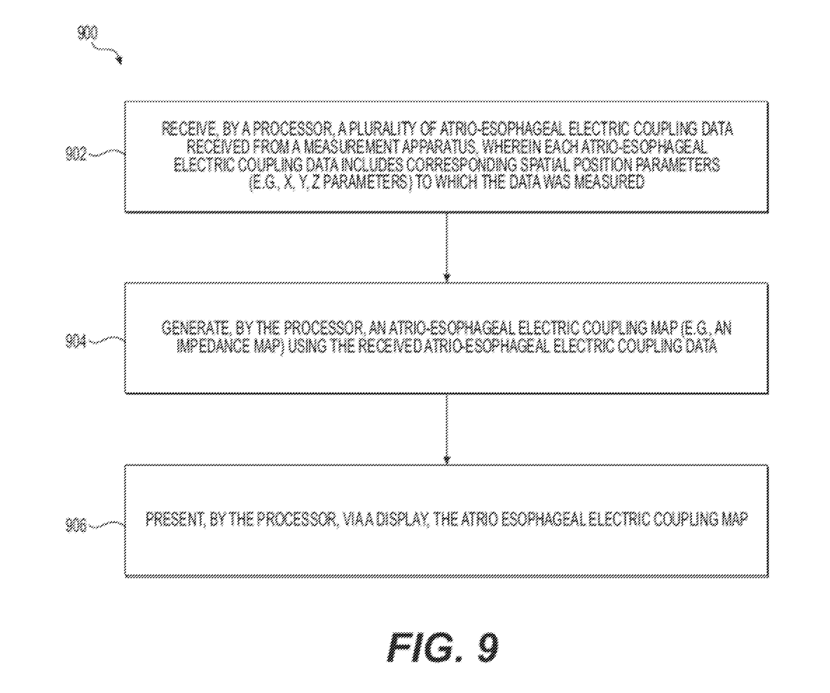

FIG. 10 depicts a diagram of an apparatus 1000 (e.g., an atrio-esophageal probe or esophageal probe), in accordance with an illustrative embodiment. The atrio-esophageal probe 1000 includes a probe body 1002 having a connector 1004 to releasably connect to one or more cables 1006 (shown as 1006a, 1006b, and 1006c).

The probe body 1002 is preferably flexible and has dimensions suitable for oral or nasal insertion into an esophagus of a patient. In some embodiments, the probe body 1002 has a dimension of about 2 cm in diameter and is at least about 10 inches long (for use for an adult patient, for example).

The apparatus 1000 includes a radio-frequency antenna 1010 coupled to the probe body 1002, wherein the radio-frequency antenna 1010 has a length 1012 that spans a region of the esophagus that substantially (e.g., greater than 90%) overlaps with the heart. In some embodiments, the length 1112 is selected from the group consisting of about 5 cm, 6 cm, 7 cm, and 8 cm. In some embodiments, the length along the esophagus is less than 5 cm. In some embodiments, the length along the esophagus is greater than 8 cm.

In some embodiments, the radio-frequency antenna 1010 has a circular cross-section area and is wound as a coil shaped structure. In some embodiments, the radio-frequency antenna has an impedance of about 50 ohms.

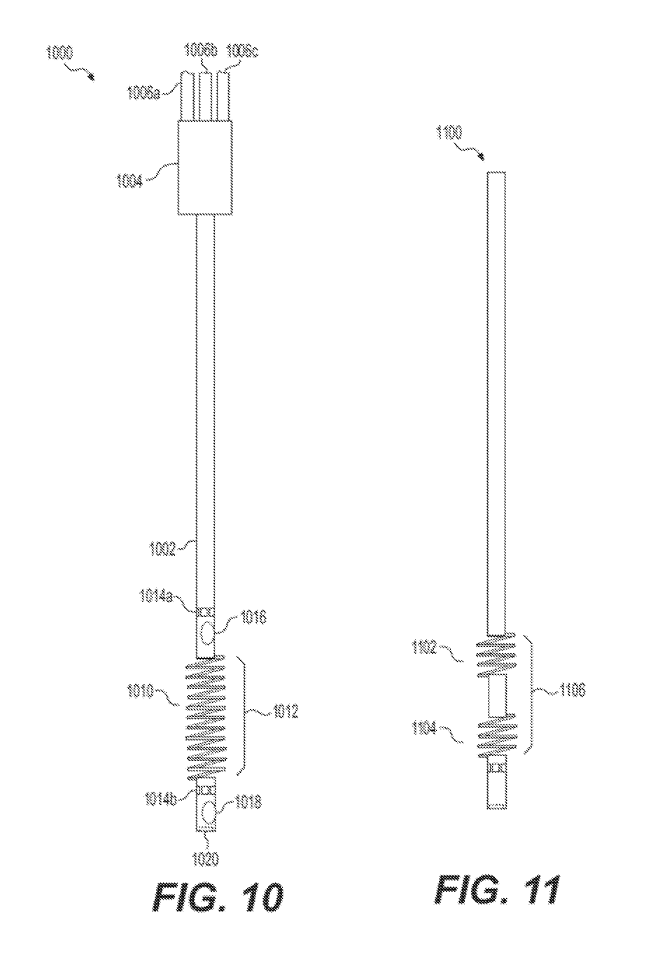

In some embodiments, the radio-frequency antenna 101 comprises a first antenna portion and a second antenna portion. FIG. 11 depicts a diagram of an apparatus 1100 (e.g., an atrio-esophageal probe or esophageal probe), in accordance with another illustrative embodiment, having a first antenna portion 1102 and a second antenna portion 1104. The first antenna portion 1102 and second antenna portion 1104 may have a length 1106 that also corresponds to a length that substantially overlaps with the heart (e.g., left atrium of the heart) when placed in the esophagus.

Referring back to FIG. 10, in some embodiments, the atrio-esophageal probe 1002 includes one or more temperature sensors 1014 (e.g., thermistor, RTDs) coupled to the probe body 1002. The temperature sensors 1014 (shown as 1014a and 1014b) may be located along the outside surface of the probe body 1002.

In some embodiments, the atrio-esophageal probe 1000 includes an irrigation port 1016 located in the probe body 1002, the irrigation port 1016 being coupled to a channel formed within the probe body 1002 to terminate at the connector 1004.

In some embodiments, the atrio-esophageal probe 1000 includes a suction port 1018 located in the probe body, the suction port 1018 being coupled to a second channel formed within the probe body 1002 to terminate at the connector 1004.

In some embodiments, the atrio-esophageal probe 1000 includes a port for an imaging transducer 1020 (e.g., ultrasound transducer, camera sensor, optical coherence tomographic transducer) to view or sample measurements from the atrio-esophageal probe 1000.

Exemplary Computer System

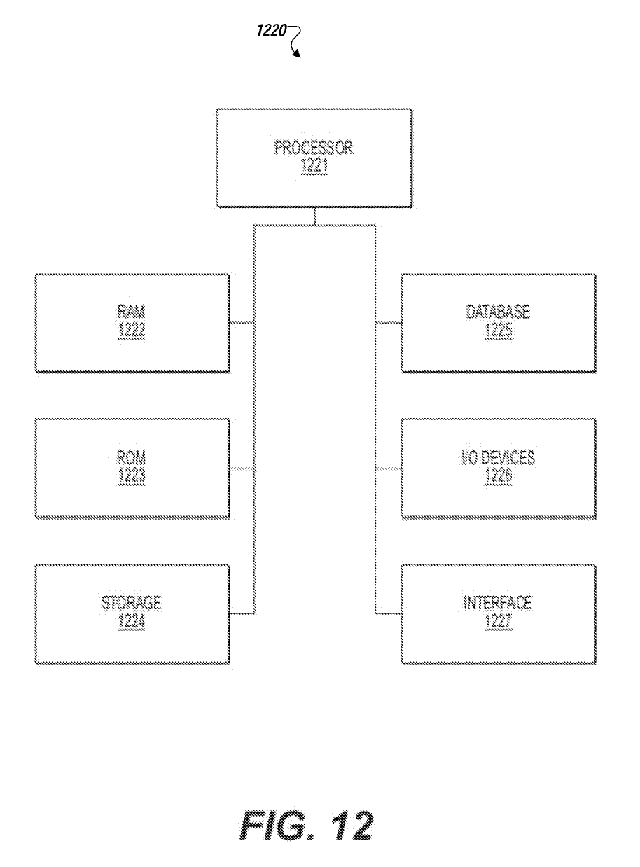

FIG. 12 illustrates an exemplary computer 1220 that can be used to generate a predictive map illustrating levels of risk of esophageal damage due to ablation of tissue in the heart, in accordance with an illustrative embodiment. As used herein, “computer” may include a plurality of computers. The computers may include one or more hardware components such as, for example, a processor 1221, a random access memory (RAM) module 1222, a read-only memory (ROM) module 1223, a storage 1224, a database 1225, one or more input/output (I/O) devices 1226, and an interface 1227. Alternatively and/or additionally, computer 1220 may include one or more software components such as, for example, a computer-readable medium including computer executable instructions for performing a method associated with the exemplary embodiments. It is contemplated that one or more of the hardware components listed above may be implemented using software. For example, storage 1224 may include a software partition associated with one or more other hardware components. It is understood that the components listed above are exemplary only and not intended to be limiting.

Processor 1221 may include one or more processors, each configured to execute instructions and process data to perform one or more functions associated with a computer for indexing images. Processor 1221 may be communicatively coupled to RAM 1222, ROM 1223, storage 1224, database 1225, I/O devices 1226, and interface 1227. Processor 1221 may be configured to execute sequences of computer program instructions to perform various processes. The computer program instructions may be loaded into RAM 1222 for execution by processor 1221. As used herein, processor refers to a physical hardware device that executes encoded instructions for performing functions on inputs and creating outputs.

RAM 1222 and ROM 1223 may each include one or more devices for storing information associated with operation of processor 1221. For example, ROM 1223 may include a memory device configured to access and store information associated with controller 1220. RAM 1222 may include a memory device for storing data associated with one or more operations of processor 1221. For example, ROM 1223 may load instructions into RAM 1222 for execution by processor 1221.

Storage 1224 may include any type of mass storage device configured to store information that processor 1221 may need to perform processes consistent with the disclosed embodiments. For example, storage 1224 may include one or more magnetic and/or optical disk devices, such as hard drives, CD-ROMs, DVD-ROMs, or any other type of mass media device.

Database 1225 may include one or more software and/or hardware components that cooperate to store, organize, sort, filter, and/or arrange data used by controller 1220 and/or processor 1221. For example, database 1225 may store hardware and/or software configuration data associated with input-output hardware devices and controllers, as described herein. It is contemplated that database 1225 may store additional and/or different information than that listed above.

I/O devices 1226 may include one or more components configured to communicate information with a user associated with controller 1220. For example, I/O devices may include a console with an integrated keyboard and mouse to allow a user to maintain a database of images, update associations, and access digital content. I/O devices 1226 may also include a display including a graphical user interface (GUI) for outputting information on a monitor. I/O devices 1226 may also include peripheral devices such as, for example, a printer for printing information associated with controller 1220, a user-accessible disk drive (e.g., a USB port, a floppy, CD-ROM, or DVD-ROM drive, etc.) to allow a user to input data stored on a portable media device, a microphone, a speaker system, or any other suitable type of interface device.

Interface 1227 may include one or more components configured to transmit and receive data via a communication network, such as the Internet, a local area network, a workstation peer-to-peer network, a direct link network, a wireless network, or any other suitable communication platform. For example, interface 1227 may include one or more modulators, demodulators, multiplexers, demultiplexers, network communication devices, wireless devices, antennas, modems, and any other type of device configured to enable data communication via a communication network.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

Throughout this application, various publications are referenced. The disclosures of these publications in their entireties are hereby incorporated by reference into this application in order to more fully describe the state of the art to which the methods and systems pertain.

It will be apparent to those skilled in the art that various modifications and variations.

1. A method for preventing esophageal injury during ablation of tissues in the heart, the method comprising:
applying, to a first electrode located within a chamber of the heart of a subject, a first set of one or more electrical signals;
measuring, from a second electrode located in the esophagus of the subject, a second set of electrical signals resulting from the first set of one or more electrical signals being applied to the first electrode, wherein the second set of electrical signals characterizes an atrio-esophageal electric coupling between the first electrode located in the chamber of the heart and the second electrode located in the esophagus; and
in response to the atrio-esophageal electric coupling, or a derivative parameter derived therefrom, satisfying an alert condition, causing an audible or visual alert to be generated.

2. The method of claim 1, comprising:
determining, by a processor, an atrio-esophageal electric coupling parameter using the measured second set of electrical signals in reference to the first set of one or more electrical signals; and
presenting, via a display, a visual representation of the determined atrio-esophageal electric coupling parameter, wherein the atrio-esophageal electric coupling parameter is expressed as an atrio-esophageal impedance.

3. (canceled)

4. The method of claim 1, wherein the first electrode is housed in an ablation apparatus and is used in the ablation of tissue in the heart.

5. The method of claim 1, wherein the first electrode is housed in an ablation apparatus, the ablation apparatus having an ablation electrode used in the ablation of tissue in the heart.

6. The method of claim 1, wherein the first set of one or more electrical signals is applied from an electric source electrically connected to the first electrode, and wherein the first set of one or more electrical signals has an oscillation frequency in a radiofrequency (RF) range.

7. The method of claim 1, wherein the second electrode comprises a conductive body having a shape and dimension suitable for oral or nasal insertion, wherein the conductive body has a length that spans a portion of the esophagus that substantially overlaps with the heart.

8. The method of claim 7, wherein the conductive body is flexible and comprises a radio-frequency antenna having a length that spans the portion of the esophagus that substantially overlaps with the heart.

9.-10. (canceled)

11. The method of claim 1 further comprising:
measuring, via a third electrode located in the esophagus of the subject, the second set of electrical signals resulting from the applied first set of one or more electrical signals, wherein the second electrode and the third electrode are mounted on a probe body to form an electrode array placed within the esophagus.

12. The method of claim 11, wherein the second and third electrodes are each located at a region, of the probe body, that substantially overlaps with the heart.

13. The method of 11 further comprising:
receiving, via one or more temperature sensors mounted on the probe body, one or more third electrical signals associated with a thermal characteristic of esophageal tissue in contact with the one or more temperature sensors; and
in response to the one or more third electrical signals, or a derivative parameter derived therefrom, satisfying a thermal alert condition, triggering the audible or visual alert.

14. The method of claim 1, comprising:
introducing, via an irrigation port located on a probe body to which the second electrode is mounted, a cooling solution into the esophagus.

15. The method of claim 1, comprising:
drawing, via a suction port located on the probe body, the introduced cooling solution from the esophagus.

16. The method of claim 1, wherein the second electrode is mounted onto a body forming a catheter.

17. The method of claim 1, wherein the step of measuring the second set of electrical signals comprises:
receiving an atrium-to-skin impedance parameter measured between a reference electrode located at a location on the skin of the subject and the first electrode; and
normalizing a parameter associated with the atrio-esophageal electric coupling with the received atrium-to-skin impedance parameter.

18. A system for preventing esophageal injury during ablation of tissue in the heart, the system comprising:
an ablation catheter;
an esophageal electrode; and
an electric meter electrically connected, via a first lead, to the ablation catheter and, via a second lead, to the esophageal electrode, the electric metering having an electric circuit configured to measure an atrio-esophageal electric coupling using a measured alternating electric signal captured at the second lead, wherein the measured alternating electric signal results from an applied alternating electric signal generated, by the electric circuit, and applied to the first lead.

19. The system of claim 18 further comprising:
a three-dimensional (3D) mapping system, the 3D mapping system being coupled to the electric meter to receive i) a first set of data associated with the atrio-esophageal electric coupling and ii) a corresponding second set of data associated with position information collected contemporaneously with the atrio-esophageal electric coupling, the three-dimensional (3D) mapping system being configured to process the first and second set of data to render, via a display, a three-dimensional representation of the atrio-esophageal electric coupling.

20. The system of claim 18, further comprising:
a vacuum, the vacuum being coupled, via a tube, to a probe body housing the esophageal electrode, the tube terminating at a suction port located at the probe body.

21. The system of claim 18 further comprising:
a pump coupled, via a second tube, to a probe body housing the esophageal electrode, the second tube terminating at an irrigation port located at the probe body.

22. The system of claim 18 further comprising:
an imaging system coupled, via a cable, to a probe body housing the esophageal electrode, the cable terminating at an imaging probe located at the probe body; and
a radiofrequency generator, the radiofrequency generator being configured to measure an atrium-to-skin impedance parameter between a reference electrode patch located at a location on the skin of the subject and the ablation catheter, the radiofrequency generator being configured to output the measured atrium-to-skin impedance parameter to be used to normalize parameters associated with the measured atrio-esophageal electric coupling.

23. (canceled)

24. A method for generating a predictive map illustrating levels of risk of esophageal damage due to ablation of tissue in the heart, the method comprising:
receiving, by a processor, a plurality of atrio-esophageal electric coupling data received from a measurement apparatus, wherein each of the atrio-esophageal electric coupling data includes corresponding spatial position parameters to which the data was measured;
generating, by the processor, an atrio-esophageal electric coupling map using the received atrio-esophageal electric coupling data, wherein the atrio-esophageal electric coupling map comprises a three-dimensional representation of the atrio-esophageal electric coupling; and
presenting, by the processor, via a display, the atrio-esophageal electric coupling map.

25.-37. (canceled)
